(19) United States
(12) Patent Application Publication
(10) Pub. No.: US 2026/0265820 A1
(43) Pub. Date: Sep. 10, 2026

(54) METHODS AND COMPOSITIONS FOR ANCHORED MULTIPLEX NGS WORKFLOWS

(71) Applicant: GUARDANT HEALTH, INC., Palo Alto, CA (US)

(72) Inventor: Andrew KENNEDY, San Diego, CA (US)

(21) Appl. No.: 19/277,876

(22) Filed: Jul. 23, 2025

Related U.S. Application Data
(60) Provisional application No. 63/675,076, filed on Jul. 24, 2024.

(51) Int. Cl. C12Q 1/6874; C12Q 1/6806; C12Q 1/6855; C12Q 1/686

(52) U.S. Cl. CPC C12Q 1/6874; C12Q 1/6806; C12Q 1/6855; C12Q 1/686

(57) ABSTRACT

Described herein are methods and compositions for analyzing nucleic acid sequences. Amplicon sequencing is particularly useful for genome targeting and detection of hot-spot mutations, copy number variations, gene fusions, InDels and single-nucleotide polymorphisms (SNPs). However, for applications such as personalized cancer monitoring, wide tracking of a plurality of variants requires use of a primer sequences of extraneous length. Methods and compositions are described herein that support reduction of the length of primers with benefits including creased purity, error reduction, reduced manufacture time and savings in cost and time if a shared primer could be applied.

### CROSS-REFERENCE TO RELATED APPLICATION

This application claims the benefit of U.S. Provisional Patent Application No. 63/675,076, filed Jul. 24, 2024, which is incorporated by reference herein in its entirety.

### BACKGROUND

Amplicons are DNA products of a polymerase chain reaction (PCR). The term amplicon is often used interchangeably with PCR product. High-throughput sequencing, also called next-generation sequencing (NGS), can be used to obtain the sequence of a PCR fragment that targets a specific genomic region. In NGS amplicon sequencing, amplicons are generated by PCR, pooled and subsequently sequenced. Since NGS-based targeted sequencing results in very high coverage of a specific region of interest, amplicon sequencing can detect variants at very low levels and frequencies. The method allows for multiplexing of samples, where hundreds of PCR fragment sequences can be determined simultaneously. These multiplexing capabilities have made amplicon sequencing efficient at covering large genomic regions. Amplicon sequencing also makes data interpretation during downstream processing more manageable in comparison to data analysis following whole genome sequencing.

Amplicon sequencing is particularly useful for genome targeting and detection of hot-spot mutations, copy number variations, gene fusions, InDels and single-nucleotide polymorphisms (SNPs). Due to its many uses, the sequencing of amplicons can be successfully applied to a variety of disciplines like nutrition, bacterial metagenomics, gene editing and clinical research.

Using AMP-seq for personalized cancer monitoring requires tracking of up to 48 variants—gene-specific primers need to be designed/ordered for each of these

Additionally, the nested gene-specific Read2-NGS primer used in the 2nd round PCR is at least 80 bp long. DNA synthesis is expensive calculated as ‘per base’, can involve further purification, can be error-prone and can be longer to manufacture.

In certain circumstances, only 20-30 bp of the 80 bp primer is unique for a patient, but a standard AMP-seq workflow involves use of the full 80 bp primer for every patient+variant combination

Strategies to reduce length of the custom long primers could lead to benefits including creased purity, error reduction, reduced manufacture time and savings in cost and time if a shared primer could be applied.

### SUMMARY OF THE INVENTION

Described herein is a method of determining the nucleotide sequence contiguous to a known target nucleotide sequence, the method comprising; hybridizing a target nucleic acid molecule comprising the known target nucleotide sequence with a population of tailed random primers; extension of a hybridized tailed random primer using the portion of the target nucleic acid molecule downstream of the site of hybridization as a template; and amplifying a portion of the target nucleic acid molecule and the tailed random primer sequence with a first tail primer and a first target-specific primer. In other embodiments, sequencing the amplified sequence (amplicon) using a first and second sequencing primer. In other embodiments, the 5′ nucleic acid sequence of the tailed random primers is identical to a first sequencing primer. In other embodiments, the first tail primer comprises a nucleic acid sequence identical to the 5′ portion of the tailed random primer. In other embodiments, the second tail primer comprises a nucleic acid sequence identical to a portion of a first sequencing primer. In other embodiments, the each tailed random primer further comprises a spacer nucleic acid sequence between the 5′ nucleic acid sequence identical or complementary to a first sequencing primer and the 3′ nucleic acid sequence comprising about 6 to about 12 random nucleotides. In other embodiments, the unhybridized primers are removed from the reaction after an extension step. In other embodiments, the second tail primer is nested with respect to the first tail primer by at least 3 nucleotides. In other embodiments, the first target-specific primer further comprises a 5′ tag sequence portion comprising a nucleic acid sequence of high GC content which is not substantially complementary to or substantially identical to any other portion of any of the In other embodiments, the second tail primer is identical to a full-length first sequencing primer. In other embodiments, the nucleic acid product is sequenced by a next-generation sequencing In other embodiments, the first and second sequencing primers are compatible with the selected next-generation sequencing method. In other embodiments, the method comprises contacting the sample, or separate portions of the sample, with a plurality of sets of first and second target-specific primers. In other embodiments, the method comprises contacting a single reaction mixture comprising the sample with a plurality of sets of first and second target-specific primers. In other embodiments, the plurality of sets of first and second target-specific primers specifically anneal to known target nucleotide sequences comprised by separate genes. In other embodiments, at least two sets of first and second target-specific primers specifically anneal to different portions of a known target nucleotide sequence. In other embodiments, at least two sets of first and second target-specific primers specifically anneal to different portions of a single gene comprising a known target nucleotide sequence. In other embodiments, at least two sets of first and second target-specific primers specifically anneal to different exons of a gene comprising a known nucleotide target sequence. In other embodiments, the plurality of first target-specific primers comprise identical 5′ tag sequence portions.

In other embodiments, the first and/or second target specific primers are truncated. In other embodiments, the first and/or second target specific primers comprise uracil and/or a modified base. In other embodiments, the first and/or second target specific primers are truncated. In other embodiments, the first and/or second target specific primers comprise uracil and/or a modified base. In other embodiments, the first and/or second sequencing primer comprise a sequence of at least partially complementary to the first and/or second target specific primers. In other embodiments, the method includes uracil excision. In other embodiments, the method includes template extension.

Further described herein is a method of determining the nucleotide sequence contiguous to a known target nucleotide sequence, the method comprising; hybridizing a target nucleic acid molecule comprising the known target nucleotide sequence with a population of tailed random primers; extension of a hybridized tailed random primer using the portion of the target nucleic acid molecule downstream of the site of hybridization as a template; amplifying a portion of the target nucleic acid molecule and the tailed random primer sequence with a first tail primer and a first target-specific primer; amplifying a portion of the amplicon with a second tail primer and a second target-specific primer; sequencing the amplified portion using a first and second sequencing primer. In various embodiments, the population of tailed random primers comprises single-stranded oligonucleotide molecules having a 5′ nucleic acid sequence identical or complementary to a first sequencing primer and a 3′ nucleic acid sequence comprising from about 6 to about 12 random nucleotides; wherein the first target-specific primer comprises a nucleic acid sequence that can specifically anneal to the known target nucleotide sequence of the target nucleic acid at the annealing temperature; wherein the second target-specific primer comprises a 3′ portion comprising a nucleic acid sequence that can specifically anneal to a portion of the known target nucleotide sequence comprised by the amplicon, and a 5′ portion comprising a nucleic acid sequence that is identical to a second sequencing primer and the second target-specific primer is nested with respect to the first target-specific primer; wherein the first tail primer comprises a nucleic acid sequence identical or complementary to all or a portion of the 5′ portion of the tailed random primer; and wherein the second tail primer comprises a nucleic acid sequence identical or complementary to a portion of the first sequencing primer and is nested with respect to the first tail primer. In other embodiments, the first and/or second target specific primers are truncated. In other embodiments, the first and/or second target specific primers comprise uracil and/or a modified base. In other embodiments, the first and/or second target specific primers are truncated. In other embodiments, the first and/or second target specific primers comprise uracil and/or a modified base. In other embodiments, the first and/or second sequencing primer comprise a sequence of at least partially complementary to the first and/or second target specific primers.

In some embodiments, the method comprises subject the sample to uracil excision. In some embodiments, the method comprises subject the sample to template extension. In some embodiments, the portions of the target-specific primers that specifically anneal to the known target will anneal specifically at a temperature of about 65° C. in a PCR buffer. In some embodiments, the sample comprises cell-free DNA (cfDNA). In some embodiments, the sample comprises genomic DNA. In some embodiments, the sample comprises RNA and the method further comprises a first step of subjecting the sample to a reverse transcriptase regimen. In some embodiments, the nucleic acids present in the sample have not been subjected to shearing or digestion. In some embodiments, the sample comprises single-stranded gDNA or cfDNA. In some embodiments, the reverse transcriptase regimen comprises the use of random hexamers. In some embodiments, a gene rearrangement comprises the known target sequence. In some embodiments, the gene rearrangement is present in a nucleic acid selected from the group consisting of: genomic DNA, RNA; and cfDNA. In some embodiments, the gene rearrangement comprises an oncogene. In some embodiments, the gene rearrangement comprises a fusion oncogene. In some embodiments, the nucleic acid product is sequenced by a next-generation sequencing method. In some embodiments, the next-generation sequencing method comprises a method selected from the group consisting of: Ion Torrent, Illumina, SOLID, 454; Massively Parallel Signature Sequencing solid-phase, reversible dye-terminator sequencing; and DNA nanoball sequencing. In some embodiments, the first and second sequencing primers are compatible with the selected next-generation sequencing method. In some embodiments, the method comprises contacting the sample, or separate portions of the sample, with a plurality of sets of first and second target-specific primers. In some embodiments, the method comprises contacting a single reaction mixture comprising the sample with a plurality of sets of first and second target-specific primers. In some embodiments, the plurality of sets of first and second target-specific primers specifically anneal to known target nucleotide sequences comprised by separate genes. In some embodiments, at least two sets of first and second target-specific primers specifically anneal to different portions of a known target nucleotide sequence. In some embodiments, at least two sets of first and second target-specific primers specifically anneal to different portions of a single gene comprising a known target nucleotide sequence. In some embodiments, at least two sets of first and second target-specific primers specifically anneal to different exons of a gene comprising a known nucleotide target sequence. In some embodiments, the plurality of first target-specific primers comprise identical 5′ tag sequence portions. In various embodiments, the method includes use of an adapter with at least partial complementarity to the first target-specific, second target-specific primer, first sequencing primer and/or second sequence primer. In various embodiments, the method includes a splint adapter. In some embodiments, each amplification step comprises a set of cycles of a PCR amplification regimen from 5 cycles to 20 cycles in length. In some embodiments, the target-specific primers and the tail primers are designed such that they will specifically anneal to their complementary sequences at an annealing temperature of from about 61 to 72° C. In some embodiments, the target-specific primers and the tail primers are designed such that they will specifically anneal to their complementary sequences at an annealing temperature of about 65° C. In some embodiments, the target nucleic acid molecule is from a sample, optionally which is a biological sample obtained from a subject. In some embodiments, the sample is obtained from a subject in need of treatment for a disease associated with a genetic alteration. In some embodiments, the disease is cancer. In some embodiments, the sample comprises a population of tumor cells. In some embodiments, the sample is a tumor biopsy. In some embodiments, the cancer is lung cancer. In some embodiments, a disease-associated gene comprises the known target sequence. In some embodiments, the target nucleic acid is a ribonucleic acid. In some embodiments, the target nucleic acid is a deoxyribonucleic acid. In some embodiments, the target nucleic acid is a messenger RNA encoded from a chromosomal segment that comprises a genetic rearrangement. In some embodiments, the target nucleic acid is a chromosomal segment that comprises a portion of a genetic rearrangement.

Further described herein is a method, comprising: contacting a nucleic acid template comprising with a plurality of different primers that share a common sequence that is 5′ to different hybridization sequences; contacting the extension product with a first tail primer and a first target-specific primer; contacting the extension product of the second step with a second tail primer and a second target-specific primer; wherein the first target-specific primer comprises a nucleic acid sequence that can specifically anneal to a known target nucleotide sequence of the target nucleic acid at the annealing temperature; wherein the second target-specific primer comprises a 3′ portion comprising a nucleic acid sequence that can specifically anneal to a portion of the known target nucleotide sequence comprised by the amplicon, and a 5′ portion comprising a nucleic acid sequence that is identical to a second sequencing primer and the second target-specific primer is nested with respect to the first target-specific primer; wherein the first tail primer comprises a nucleic acid sequence identical or complementary to the common sequence of the primers of the plurality of different primers; and wherein the second tail primer comprises a nucleic acid sequence identical or complementary to a portion of the first sequencing primer and is nested with respect to the first tail primer. In various embodiment, the method includes use of conditions to achieve the specified step (e.g., template-specific hybridization and extension from the first tail primer and first target-specific primer, template-specific hybridization and extension of at least one of the plurality of different primers, template-specific hybridization and extension from the first tail primer and first target-specific primer, template-specific hybridization and extension from the second tail primer and second target-specific primer. In other embodiments, the first and/or second target specific primers are truncated. In other embodiments, the first and/or second target specific truncated primers are less than 70, less than 60, less than 50, less than 40, less than 30, less than 20 base pairs. In other embodiments, the first and/or second target specific primers each comprise uracil and/or a modified base. In other embodiments, the first and/or second target specific primers comprise uracil and/or a modified base. In other embodiments, the first and/or second sequencing primer comprise a sequence of at least partially complementary to the first and/or second target specific primers.

In some embodiments, the target nucleic acid is a ribonucleic acid. In some embodiments, the target nucleic acid is a deoxyribonucleic acid, including cell-free DNA. In some embodiments, the target nucleic acid is a messenger RNA encoded from a chromosomal segment that comprises a genetic rearrangement. In some embodiments, the target nucleic acid is a chromosomal segment that comprises a portion of a genetic rearrangement. In some embodiments, the genetic rearrangement is an inversion, deletion, or translocation. In some embodiments, the method further comprises amplifying one or more of the extension products In some embodiments, each of the primers of the first step further comprises a spacer nucleic acid sequence between the common sequence and the hybridization sequence, the spacer sequence comprising about 6 to about 12 random nucleotides. In some embodiments, the method includes uracil excision. In some embodiments, the method includes template extension. In some embodiments, the unhybridized primers are removed from the reaction after extension. In various embodiments, the uracil excision is performed prior to sequencing. In various embodiments, the uracil excision is performed on a final sequencing library. In various embodiments, the uracil excision generates an overhang. In various embodiments, the overhang. is 2, 3, 4, 5, 6, 7, 8, 9 and/or 10 or more bases. In various embodiments, the overhang includes, 3, 4, 5, 6, 7, 8, 9 and/or 10 or more base pairs in a sequence at least partially complementary to a first target-specific primer and/or second target-specific primer. In various embodiments, the method includes use of an adapter with at least partial complementarity to the first target-specific, second target-specific primer, first sequencing primer and/or second sequence primer. In various embodiments, the method includes a splint adapter.

In some embodiments, the second tail primer is nested with respect to the first tail primer by at least 3 nucleotides. In some embodiments, the first target-specific primer further comprises a 5′ tag sequence portion comprising a nucleic acid sequence of high GC content which is not substantially complementary to or substantially identical to any other portion of any of the primers. In some embodiments, the portions of the target-specific primers that specifically anneal to the known target will anneal specifically at a temperature of about 65° C. in a PCR buffer.

### BRIEF DESCRIPTION OF THE DRAWINGS

FIG. 1. Representative workflow of existing, conventional methods.

FIG. 2. Read2 overhang dsDNA adapter ligation. FIG. 2A. Read2 overhang dsDNA adapter ligation, for example dsDNA Read2-adapter ligation. FIG. 2A. Oligo end blocking to mitigate undesired ligation events.

FIG. 3. Read2 overhang dsDNA adapter ligation. FIG. 3A. As depicted, shown is Read2 overhang dsDNA adapter ligation includes dsDNA Read2-adapter ligation. FIG. 3B. P5-primer overhang to mitigate undesired ligation events, such as the depicted 5′ tail containing uracial (e.g., polyA).

FIG. 4. Truncated Read1-adapter ligation. FIG. 4A. As depicted, shown is Truncated Read1-adapter ligation variant compatible with all workflows above. FIG. 4B. Read2 overhang dsDNA adapter ligation includes dsDNA Read2-adapter ligation.

FIG. 5. Read1- and Read2-overhang adapter workflow. FIG. 5A. As shown, depicted is Read1- and Read2-overhang adapter workflow (dsDNA). FIG. 5B. Depicted are overhangs target asymmetric sequence ends of Read1- and Read2-primers.

FIG. 6. Read1- and Read2-splint adapter workflow. FIG. 6A. Read1- and Read2-splint adapter workflow. FIG. 6B. Shown are overhangs that target asymmetric sequence ends of Read1- and Read2-primers.

FIG. 7. Common Read2-primer extension includes Common Read2-primer extension. FIG. 7A. As shown, depicted compatibility with full and truncated Read1-side adapter ligation workflow. FIG. 7B. templated extension can be cycled to give multiple extensions per template molecule-requiring lower template oligo than PCR.

FIG. 8. Long primer manufacture from gene-specific custom primers.

### DETAILED DESCRIPTION

As described, AmpSeq uses two rounds of PCR: a 1st PCR to amplify a multiplex of markers and incorporate linker sequences, and a 2nd PCR to use the linker sequences to add a unique pair of indices, or barcodes, to each sample. Adapters for forward and reverse primer can include linker sequences added to the 5′ end of each reverse primer to accommodate barcode adapters.

Briefly, double-stranded cDNA undergoes end repair, adenylation and ligation, with a universal half-functional adapter. The resulting half-functional library by itself is insufficient for downstream bridge amplification, emulsion PCR or sequencing. The library is rendered fully functional at the end of two rounds of nested low-cycle PCR, which represent the core steps for target enrichment. The second round of PCR uses nested primers that are 5′ tagged with a common sequencing adapter. In combination with the first half-functional universal adapter, the resulting target amplicons are functionalized for clonal amplification (for example, emulsion PCR or bridge PCR) and sequencing. Nontarget fragments remain half-functional (inconsequential) and need not be eliminated from the library. Typically, in conventional methods, libraries are quantitated and processed for sequencing.

However, here, adapters are adjusted to include uracil and/or other modified bases, which following second PCR are then subject to excision, leaving an overhang which can then be utilized for adaptation of a common Read2-NGS overhang adapter for subsequent sequencing. Importantly, this allows for introduction of a truncated adapter rather than the longer primers of typical nested target gene-specific and further use of the common overhang adapter.

AMP-seq has been described as a rapid enrichment method for targeted RNA and DNA next-generation sequencing. While useful, the existing approach relies on a core set of standard molecular biology reagents, which subsequently utilizes primers that may be quickly designed and synthesized as part of a facile, custom targeted sequencing solution requiring library construction, which expensive and ineffective by relying on longer primer sequence. Without being bound by any particular hypothesis the shorter provided sequences and universal nature of the manipulation steps is likely to support better efficiency to improve sensitivity of detection of gene fusions, point mutations, insertions, deletions and copy number changes

### Ligation of Adapters

Double-stranded nucleic acids e.g., DNA molecules in a sample, and single stranded nucleic acid molecules converted to double stranded molecules, can be linked to adapters at either one end or both ends. In the methods of the disclosure, adapters can be ligated to sample nucleic acids prior to the partitioning and/or conversion steps. In some embodiments, adapters may be ligated to the sample nucleic acids after the partitioning and conversion steps, but before the step of amplifying the nucleic acids which have been subjected to partitioning and conversion steps.

In some embodiments, the DNA is made ligatable, e.g., by extending the end overhangs of the DNA molecules, and adding adenosine residues to the 3′ ends of fragments and phosphorylating the 5′ end of each DNA fragment. Typically, double stranded molecules are blunt ended by treatment with a polymerase with a 5′-3′ polymerase and a 3′-5′ exonuclease (or proof reading function), in the presence of all four standard nucleotides. Klenow large fragment and T4 polymerase are examples of suitable polymerase.

The blunt ended DNA molecules can be ligated with at least partially double stranded adapter (e.g., a Y shaped or bell-shaped adapter). Alternatively, complementary nucleotides can be added to blunt ends of sample nucleic acids and adapters to facilitate ligation. Contemplated herein are both blunt end ligation and sticky end ligation. In blunt end ligation, both the sample nucleic acid molecules and the adapters have blunt ends. In sticky-end ligation, typically, the sample nucleic acid molecules bear an “A” overhang and the adapters bear a “T” overhang.

DNA ligase and adapters are added to ligate DNA molecules in the sample with an adapter on one or both ends, i.e. to form adapted DNA. As used herein, “adapter” refers to short nucleic acids (e.g., less than about 500, less than about 100 or less than about 50 nucleotides in length, or be 20-30, 20-40, 30-50, 30-60, 40-60, 40-70, 50-60, 50-70, 20-500, or 30-100 bases from end to end) that are typically at least partially double-stranded and can be ligated to the end of a given sample nucleic acid molecule. In some instances, two adapters can be ligated to a single sample nucleic acid molecule, with one adapter ligated to each end of the sample nucleic acid molecule.

Adapters can include nucleic acid primer binding sites to permit amplification of a sample nucleic acid molecule flanked by adapters at both ends, and/or a sequencing primer binding site, including primer binding sites for sequencing applications, such as various next generation sequencing (NGS) applications. Adapters can include a sequence for hybridizing to a solid support, e.g., a flow cell sequence. Adapters can also include binding sites for capture probes, such as an oligonucleotide attached to a flow cell support or the like. Adapters can also include sample indexes and/or molecular barcodes. These are typically positioned relative to amplification primer and sequencing primer binding sites, such that the sample index and/or molecular barcode is included in amplicons and sequencing reads of a given nucleic acid molecule. Adapters of the same or different sequence can be linked to the respective ends of a sample nucleic acid molecule. In some embodiments, adapters of the same or different sequence are linked to the respective ends of the nucleic acid molecule except that the sample index and/or molecular barcode differs in its sequence. In some embodiments, the adapter is a Y-shaped adapter in which one end is blunt ended or tailed as described herein, for joining to a nucleic acid molecule, which is also blunt ended or tailed with one or more complementary nucleotides to those in the tail of the adapter. In another exemplary embodiment, an adapter is a bell-shaped adapter that includes a blunt or tailed end for joining to a nucleic acid molecule to be analyzed. Other exemplary adapters include T-tailed, C-tailed or hairpin shaped adapters. For example, a hairpin shaped adapter can comprise a complementary double stranded portion and a loop portion, where the double stranded portion can be attached (e.g., ligated) to a double-stranded polynucleotide. Hairpin shaped sequencing adapters can be attached to both ends of a polynucleotide fragment to generate a circular molecule, which can be sequenced multiple times.

In some embodiments, the nucleic acids further comprise adapters in which at least one cytosine is a modification resistant cytosine, optionally wherein each cytosine in the adapters is a modification resistant cytosine. In some embodiments, methods further comprise further comprising ligating adapters to the nucleic acids, wherein at least one cytosine in the adapters is a modification resistant cytosine, optionally wherein the ligating occurs before step (c) and/or after step (a); further optionally wherein each cytosine in the adapters is a modification resistant cytosine. The adapters may comprise barcodes, e.g., according to any of the embodiments relating to barcodes described elsewhere herein. In some embodiments, the adapters can include at least 1, 2, 3, 4, 5, 6, 7, 8, 9, 10, 11, 12, 13, 14, 15, 16, 17, 18, 19, or 20 modified nucleotides, such as modified cytosine nucleotides, that are resistant to modification, e.g., conversion. In some embodiments, the modified nucleotides are resistant to modification by a deaminase. In some embodiments, the modified nucleotides comprise a conversion resistant modified cytosine, such as 5-propynylC (5pyC), 5-pyrrolo-dC (5pyrC), 5-hydroxymethylcytosine (5hmC) along with modified variants thereof, glucosylated5-hydroxymethylcytosine (5ghmC), cytosine 5-methylenesulfonate (CMS), bulky 5-position adducts, or N4-modified cytosine. In some embodiments, the conversion resistant modified cytosine is 5pyC, 5pyrC, 5ghmC, or CMS. In some embodiments, the conversion resistant modified cytosine can protect cytosine from being converted by a deaminase, such as a cytidine deaminase, which converts a cytosine to uracil. In some embodiments, each cytosine of an adapter is a conversion resistant modified cytosine, such as any one or more of the foregoing examples. For exemplary descriptions of modified nucleotides and their use in adaptors, see WO2023/288222 and U.S. Pat. No. 10,260,088.

The adapters used in the methods of the present disclosure may comprise one or more known nucleosides wherein the base has a known methylation status, such as 5mC nucleic acid bases. When using adapters comprise 5mC, the adapters can be ligated to the sample nucleic acid molecules prior to the conversion procedure. Analyzing the sequence data corresponding to these known 5mC nucleic acid bases allows for the efficiency of the conversion procedure to be measured, which can be used as a quality control measure for the conversion procedure. In instances where two adapters are ligated to a sample nucleic acid (one at each end), either or both of the adapters may comprise one or more nucleosides with a known methylation status. Typically the primer binding site(s), sequencing primer binding site(s), sample index(es) and/or molecular barcode(s), if present, do not comprise the nucleosides with a methylation status that change base pairing specificity as a result of the conversion procedure.

Preferably adapters (e.g., Y-shaped adapters) are ligated to the sample nucleic acids prior to the conversion and partitioning steps.

In some embodiments, the disclosed methods comprise analyzing DNA in a sample. In such methods, adapters may be added to the DNA. This may be done concurrently with an amplification procedure, e.g., by providing the adapters in a 5′ portion of a primer (where PCR is used, this can be referred to as library prep-PCR or LP-PCR), before, or after an amplification step. In some embodiments, adapters are added by other approaches, such as ligation. In some such methods, first adapters are added to the 3′ ends of the nucleic acids by ligation, which may include ligation to single-stranded DNA. In some such methods, first adapters are added to the 5′ ends of the nucleic acids by ligation, which may include ligation to single-stranded DNA. In some embodiments, prior to any partitioning or capturing steps, first adapters are added to the nucleic acids by ligation, which may include ligation to single-stranded DNA (e.g., to the 3′ ends thereof). In some embodiments, the capture probes can be isolated after partitioning and ligation. For example, the hypomethylated partition can be ligated with adapters and a portion of the ligated hypomethylated partition can then be used to generate the capture probes for rearrangements. The adapter can be used as a priming site for second-strand synthesis, e.g., using a universal primer and a DNA polymerase. A second adapter can then be ligated to at least the 3′ end of the second strand of the now double-stranded molecule. In some embodiments, the first adapter includes an affinity tag, such as biotin, and nucleic acid ligated to the first adapter is bound to a solid support (e.g., bead), which may comprise a binding partner for the affinity tag such as streptavidin. For further discussion of a related procedure, see Gansauge et al., Nature Protocols 8:737-748 (2013). Commercial kits for sequencing library preparation compatible with single-stranded nucleic acids are available, e.g., the Accel-NGS® Methyl-Seq DNA Library Kit from Swift Biosciences. In some embodiments, after adapter ligation, nucleic acids are amplified.

In some embodiments, the single-stranded DNA library preparation is performed in a one-step combined phosphorylation/ligation reaction, e.g., as described in Troll et al., BMC Genomics, 20:1023 (2019), available at doi.org/10.1186/s12864-019-6355-0. This method, called Single Reaction Single-stranded LibrarY (“SRSLY,”) can be performed without end-polishing. SRSLY may be useful for converting short and fragmented DNA molecules, e.g., cfDNA fragments, into sequencing libraries while retaining native lengths and ends. The SRSLY method can create sequencing libraries (e.g., Illumina sequencing libraries) from fragmented or degraded template (input) DNA. In particular embodiments, template DNA is first heat denatured and then immediately cold shocked to render the template DNA molecules single-stranded. The DNA can be maintained as single-stranded throughout the ligation reaction by the inclusion of a thermostable single-stranded binding protein (SSB). Next, the template DNA, which at this point can be single-stranded and coated with SSB, is placed in a phosphorylation/ligation dual reaction with directional dsDNA NGS adapters that contain single-stranded overhangs. Both the forward and reverse sequencing adapters can share similar structures but differ in which termini is unblocked in order to facilitate proper ligations. Both sequencing adapters can comprise a dsDNA portion and a single-stranded splint overhang of random nucleotides that occurs on the 3-prime terminus of the bottom strand of the forward adapter and the 5-prime terminus of the bottom strand of the reverse adapter. In this way, the forward adapter (e.g., (P5) Illumina adapter) can delivered to the 5-prime end of template molecules and the reverse adapter (e.g., (P7) Illumina adapter) is delivered to the 3-prime end of template molecules. Thus, the native polarity of input DNA molecules can be retained.

During the dual phosphorylation/ligation reaction, T4 Polynucleotide Kinase (PNK) can be used to prepare template DNA termini for ligation by phosphorylating 5-prime termini and dephosphorylating 3-prime termini. T4 PNK works on both ssDNA and dsDNA molecules and has no activity on the phosphorylation state of proteins. Simultaneously, the random nucleotides of the splint adapter can be annealed to the single-stranded template molecule. This creates a short, localized dsDNA molecule, enabling ligation of template to adapter with a ligase such as T4 DNA ligase, which has high ligation efficiency on dsDNA templates but low efficiency on ssDNA. After the single phosphorylation/ligation reaction is complete, the library DNA can be, e.g., purified and placed directly into standard NGS indexing PCR, compatible with both traditional single or dual index primers.

### Molecular Tagging

In some embodiments, the nucleic acid molecules of the sample may be tagged with sample indexes, partition tags and/or molecular barcodes (referred to generally as “tags”). Tags can form part of an adapter.

Tags can be molecules, such as nucleic acids, containing information that indicates a feature of the molecule with which the tag is associated. For example, molecules can bear a sample tag or sample index (which distinguishes molecules in one sample from those in a different sample), a partition tag (which distinguishes molecules in one partition from those in a different partition) and/or a molecular tag/molecular barcode/barcode (which distinguishes different molecules from one another (in both unique and non-unique tagging scenarios). In certain embodiments, a tag can comprise one or a combination of barcodes. As used herein, the term “barcode” refers to a nucleic acid molecule having a particular nucleotide sequence, or to the nucleotide sequence, itself, depending on context. A barcode can have, for example, between 10 and 100 nucleotides. A collection of barcodes can have degenerate sequences or can have sequences having a certain Hamming distance, as desired for the specific purpose. So, for example, a molecular barcode can be comprised of one barcode or a combination of two barcodes, each attached to different ends of a molecule. Additionally or alternatively, for different partitions and/or samples, different sets of molecular barcodes, molecular tags, or molecular indexes can be used such that the barcodes serve as a molecular tag through their individual sequences and also serve to identify the partition and/or sample to which they correspond based the set of which they are a member. For example, barcodes can be used to allow the origin of the DNA (e.g., the subject, biological sample (e.g., samples collected at various time points), enriched DNA sample (e.g., enriched DNA comprising an epigenetic target region set or enriched DNA comprising a sequence-variable target region set), partition, or similar) to be identified, e.g., following pooling of a plurality of samples for parallel sequencing.

In the methods of the disclosure, partitioning results in the generation of multiple subsamples (i.e. partitions) based on the presence or absence of 5hmC nucleic acid bases in the sample nucleic acids. Tags can be used to label the nucleic acids in each partition so as to correlate the tag (or tags) with a specific partition. For example, if multiple subsamples are carried forward after the partitioning step, tags can be used to label each of the subsamples such that the corresponding sequence data deriving from each subsample can be identified. In some embodiments, a single tag can be used to label a specific partition. In some embodiments, multiple different tags can be used to label a specific partition. In embodiments employing multiple different tags to label a specific partition, the set of tags used to label one partition can be readily differentiated for the set of tags used to label other partitions. In some embodiments, the tags may have additional functions, for example the tags can be used to index sample sources or used as unique molecular identifiers (which can be used to improve the quality of sequencing data by differentiating sequencing errors from mutations, for example as in Kinde et al., Proc Nat'l Acad Sci USA 108: 9530-9535 (2011), Kou et al., PLOS ONE, 11: e0146638 (2016)) or used as non-unique molecule identifiers, for example as described in U.S. Pat. No. 9,598,731. Similarly, in some embodiments, the tags may have additional functions, for example the tags can be used to index sample sources or used as non-unique molecular identifiers (which can be used to improve the quality of sequencing data by differentiating sequencing errors from mutations).

Tags may be incorporated into or otherwise joined to adapters by chemical synthesis, ligation (e.g., as described above, e.g., by blunt-end ligation or sticky-end ligation), or overlap extension polymerase chain reaction (PCR), among other methods. Such adapters are ultimately joined to the target nucleic acid molecule. In other embodiments, one or more rounds of amplification cycles (e.g., PCR amplification) may be applied to introduce sample indexes to a nucleic acid using conventional nucleic acid amplification methods. The amplifications may be conducted in one or more reaction mixtures (e.g., a plurality of microwells in an array). Molecular barcodes and/or sample indexes may be introduced simultaneously, or in any sequential order. In some embodiments, molecular barcodes and/or sample indexes are introduced prior to and/or after the conversion procedure. In some embodiments, molecular barcodes and/or sample indexes are introduced prior to and/or after the partitioning step. In some embodiments, molecular barcodes and/or sample indexes are introduced prior to and/or after sequence capturing steps, if present, are performed. In some embodiments, only the molecular barcodes are introduced prior to probe capturing and the sample indexes are introduced after sequence capturing steps are performed. In some embodiments, both the molecular barcodes and the sample indexes are introduced prior to performing probe-based sequence capturing steps, if present. In some embodiments, the sample indexes are introduced after sequence capturing steps are performed, if present. In some embodiments, sample indexes are incorporated through overlap extension polymerase chain reaction (PCR).

In some embodiments, the tags may be located at one end or at both ends of the sample nucleic acids. In some embodiments, tags are predetermined or random or semi-random sequences. In some embodiments, the tag(s) may together be less than about 500, 200, 100, 50, 20, 10, 9, 8, 7, 6, 5, 4, 3, 2, or 1 nucleotide in length. Typically, tags are about 5 to 20 or 6 to 15 nucleotides in length. The tags may be linked to sample nucleic acids randomly or non-randomly.

In some embodiments, each sample is uniquely tagged with a sample index or a combination of sample indexes. In some examples, when multiple subsamples (i.e. partitions) are subsequently processed after the partitioning step, each partition can be uniquely tagged with a partition tag or a combination of partition tags. In some embodiments, each nucleic acid molecule of a sample or subsample is uniquely tagged with a molecular barcode or a combination of molecular barcodes. In other embodiments, a plurality of molecular barcodes may be used such that molecular barcodes are not necessarily unique to one another in the plurality (e.g., non-unique molecular barcodes). In these embodiments, molecular barcodes are generally attached (e.g., by ligation) to individual nucleic acid molecules such that the combination of the molecular barcode and the sequence of the sample nucleic acid that it is attached to creates a unique sequence that may be individually tracked. Detection of non-unique molecular barcodes in combination with endogenous sequence information typically allows for the assignment of a unique identity to a particular molecule. Endogenous sequence information includes the beginning (start) and/or end (stop) genomic location/position corresponding to the sequence of the original nucleic acid molecule in the sample, start and stop genomic positions corresponding to the sequence of the original nucleic acid molecule in the sample, the beginning (start) and/or end (stop) genomic location/position of the sequence read that is mapped to the reference sequence, start and stop genomic positions of the sequence read that is mapped to the reference sequence, sub-sequences of sequence reads at one or both ends, length of sequence reads, and/or length of the original nucleic acid molecule in the sample. In some embodiments, beginning region comprises the first 1, first 2, the first 5, the first 10, the first 15, the first 20, the first 25, the first 30 or at least the first 30 base positions at the 5′ end of the sequencing read that align to the reference sequence. In some embodiments, the end region comprises the last 1, last 2, the last 5, the last 10, the last 15, the last 20, the last 25, the last 30 or at least the last 30 base positions at the 3′ end of the sequencing read that align to the reference sequence. The length, or number of base pairs, of an individual sequence read are also optionally used to assign a unique identity to a given molecule. As described herein, fragments from a single strand of nucleic acid having been assigned a unique identity, may thereby permit subsequent identification of fragments from the parent strand, and/or a complementary strand.

In certain embodiments, the number of different tags used to uniquely identify a number of molecules, z, in a class can be between any of 2*z, 3*z, 4*z, 5*z, 6*z, 7*z, 8*z, 9*z, 10*z, 11*z, 12*z, 13*z, 14*z, 15*z, 16*z, 17*z, 18*z, 19*z, 20*z or 100*z (e.g., lower limit) and any of 100,000*z, 10,000*z, 1000*z or 100*z (e.g., upper limit). In some embodiments, molecular barcodes are introduced at an expected ratio of a set of identifiers (e.g., a combination of unique or non-unique molecular barcodes) to molecules in a sample. One example format uses from about 2 to about 1,000,000 different molecular barcode sequences, or from about 5 to about 150 different molecular barcode sequences, or from about 20 to about 50 different molecular barcode sequences, ligated to both ends of a target molecule. Alternatively, from about 25 to about 1,000,000 different molecular barcode sequences may be used. For example, 20-50×20-50 molecular barcode sequences (i.e., one of the 20-50 different molecular barcode sequences can be attached to each end of the target molecule) can be used. Such numbers of identifiers are typically sufficient for different molecules having the same start and stop points to have a high probability (e.g., at least 94%, 99.5%, 99.99%, or 99.999%) of receiving different combinations of identifiers.

In some embodiments, the assignment of unique or non-unique molecular barcodes in reactions is performed using methods and systems described in, for example, U.S. Patent Application Nos. 20010053519, 20030152490, and 20110160078, and U.S. Pat. Nos. 6,582,908, 7,537,898, 9,598,731, and 9,902,992, each of which is hereby incorporated by reference in its entirety. Alternatively, in some embodiments, different nucleic acid molecules of a sample may be identified using only endogenous sequence information (e.g., start and/or stop positions, sub-sequences of one or both ends of a sequence, and/or lengths). The addition of tags (e.g., sample indexes, partition tags and/or molecular barcodes) to nucleic acids can be done through amplification, wherein the tags are comprised in primers used for amplification.

In some embodiments, the nucleic acids are ligated to adapters comprising molecular barcodes. These molecular barcodes (optionally in combination with endogenous sequence information) can then be used when analyzing the sequencing data to group sequence reads deriving from the same parent nucleic acids (i.e. those nucleic acids prior to any amplification). The grouped sequence reads can then be analyzed, for example, to determine a consensus sequence for parent nucleic acids. The consensus sequence will include any converted bases and thus can be used to determine the methylation status of the parent nucleic acid. Similarly, the abundance of consensus sequences from a subsample at C positions in a reference can be used to determine the 5hmC status of the parent nucleic acids. For instance, when the base coverage of a specific C position in a reference sequence is higher than other C positions in a subsample which has been enriched for 5hmC, that specific C position on that parent nucleic acid can be identified as comprising a 5hmC modification at that C position.

### Capturing Using Capture Probes

Nucleic acids in a sample can be subject to a sequence capture step, in which molecules having target sequences are captured for subsequent analysis. Capture may be performed using any suitable approach known in the art. Target capture can involve use of a bait set comprising oligonucleotide baits labeled with a capture moiety, such as biotin or the other examples noted below. The probes can have sequences selected to tile across a panel of regions, such as genes. Such bait sets are combined with a sample under conditions that allow hybridization of the target molecules with the baits. Then, captured molecules are isolated using the capture moiety. For example, a biotin capture moiety by bead-based streptavidin. Such methods are further described in, for example, U.S. Pat. No. 9,850,523, issuing Dec. 26, 2017, which is incorporated herein by reference.

Capture moieties include, without limitation, biotin, avidin, streptavidin, a nucleic acid comprising a particular nucleotide sequence, a hapten recognized by an antibody, and magnetically attractable particles. The extraction moiety can be a member of a binding pair, such as biotin/streptavidin or hapten/antibody. In some embodiments, a capture moiety that is attached to an analyte is captured by its binding pair which is attached to an isolatable moiety, such as a magnetically attractable particle or a large particle that can be sedimented through centrifugation. The capture moiety can be any type of molecule that allows affinity separation of nucleic acids bearing the capture moiety from nucleic acids lacking the capture moiety. Exemplary capture moieties are biotin which allows affinity separation by binding to streptavidin linked or linkable to a solid phase or an oligonucleotide, which allows affinity separation through binding to a complementary oligonucleotide linked or linkable to a solid phase.

In some embodiments, the methods herein comprise capturing nucleic acids comprising epigenetic and/or sequence-variable target regions. Such regions may be captured from a sample (e.g., a subsample) that has undergone attachment of adapters, conversion, partitioning, and/or amplification). Enriching for or capturing DNA comprising epigenetic and/or sequence-variable target regions may comprise contacting the DNA with a set of target-specific probes. The set of target-specific probes may have any of the features described herein for sets of target-specific probes, including but not limited to in the embodiments set forth above and the sections relating to probes below. Capturing may be performed on one or more subsamples prepared during methods disclosed herein. In some embodiments, DNA is captured from the first subsample and/or the second subsample, e.g., the first subsample and the second subsample. In some embodiments, the subsamples are differentially tagged (e.g., as described herein) and then pooled before undergoing capture.

The capturing step may be performed using conditions suitable for specific nucleic acid hybridization, which generally depend to some extent on features of the probes such as length, base composition, etc. Those skilled in the art will be familiar with appropriate conditions given general knowledge in the art regarding nucleic acid hybridization. In some embodiments, complexes of target-specific probes and DNA are formed.

In some embodiments, methods described herein comprise capturing a plurality of sets of target regions of cfDNA obtained from a subject (e.g., test subject). The target regions comprise intronic regions or VDJ regions that may comprise rearrangements, epigenetic target regions, which may show differences in methylation levels and/or fragmentation patterns depending on whether they originated from a tumor or from healthy cells, and sequence-variable regions, which may show differences in sequence, other than rearrangements, depending on whether they originated from a tumor or from healthy cells. The capturing step produces a captured set of cfDNA molecules. In some embodiments, the cfDNA molecules corresponding to the sequence-variable target region set are captured at a greater capture yield in the captured set of cfDNA molecules than cfDNA molecules corresponding to the epigenetic target region set. In some embodiments, a method described herein comprises contacting cfDNA obtained from a subject (e.g., a test subject) with a set of target-specific probes, wherein the set of target-specific probes is configured to capture cfDNA corresponding to the sequence-variable target region set at a greater capture yield than cfDNA corresponding to the epigenetic target region set. For additional discussion of capturing steps, capture yields, and related aspects, see WO2020/160414, which is incorporated herein by reference for all purposes.

It can be beneficial to capture cfDNA corresponding to the sequence-variable target region set at a greater capture yield than cfDNA corresponding to the epigenetic target region set because a greater depth of sequencing may be necessary to analyze the sequence-variable target regions with sufficient confidence or accuracy than may be necessary to analyze the epigenetic target regions. The volume of data needed to determine fragmentation patterns (e.g., to test for perturbation of transcription start sites or CTCF binding sites) or methylation status is generally less than the volume of data needed to determine the presence or absence of cancer-related sequence mutations. Capturing the target region sets at different yields can facilitate sequencing the target regions to different depths of sequencing in the same sequencing run (e.g., using a pooled mixture and/or in the same sequencing cell).

In some embodiments, amplification is performed before the capturing step. In some embodiments, amplification is performed after the capturing step. In some embodiments, amplification is performed before and after the capturing step. In some embodiments, the methods further comprise sequencing the captured cfDNA to different degrees of sequencing depth for the epigenetic and sequence-variable target region sets and for rearrangements, consistent with the discussion herein.

In some embodiments, a capturing step is performed with probes for a sequence-variable target region set and probes for an epigenetic target region set in the same vessel at the same time, e.g., the probes for the sequence-variable and epigenetic target region sets are in the same composition. This approach provides a relatively streamlined workflow. In some embodiments, the concentration of the probes for the sequence-variable target region set is greater that the concentration of the probes for the epigenetic target region set.

Alternatively, a capturing step is performed with a sequence-variable target region probe set in a first vessel and with an epigenetic target region probe set in a second vessel, or a contacting step is performed with a sequence-variable target region probe set at a first time and a first vessel and an epigenetic target region probe set at a second time before or after the first time. This approach allows for preparation of separate first and second compositions comprising captured DNA corresponding to a sequence-variable target region set and captured DNA corresponding to an epigenetic target region set. The compositions can be processed separately as desired (e.g., to partition based on methylation as described herein). These can then be pooled in appropriate proportions to provide material for further processing and analysis such as sequencing.

### Sequencing

In general, sample nucleic acids flanked by adapters can be subject to sequencing after amplification. Sequencing methods include, for example, Sanger sequencing, high-throughput sequencing, pyrosequencing, sequencing-by-synthesis, single-molecule sequencing (also known as long-read sequencing or third generation sequencing), nanopore sequencing (a type of long-read sequencing), 5-letter sequencing or 6-letter sequencing, semiconductor sequencing, sequencing-by-ligation, sequencing-by-hybridization, Digital Gene Expression (Helicos), next generation sequencing (NGS), Single Molecule Sequencing by Synthesis (SMSS) (Helicos), enzymatic methyl sequencing (EM-Seq), Tet-assisted pyridine borane sequencing (TAPS), massively-parallel sequencing, Clonal Single Molecule Array (Solexa), shotgun sequencing, Ion Torrent, Oxford Nanopore, Roche Genia, Maxim-Gilbert sequencing, primer walking, and sequencing using PacBio, SOLiD, Ion Torrent, or Nanopore platforms. Sequencing reactions can be performed in a variety of sample processing units, which may include multiple lanes, multiple channels, multiple wells, or other means of processing multiple sample sets substantially simultaneously. Sample processing unit can also include multiple sample chambers to enable processing of multiple runs simultaneously. For example, long-read sequencing (also referred to herein as single-molecule sequencing or third generation sequencing) methods include those that can generate longer sequencing reads, such as reads in excess of 10 kilobases, as compared to short-read sequencing methods, which generally produce reads of up to about 600 bases in length. Compared to short reads, long reads can improve de novo assembly, transcript isoform identification, and detection and/or mapping of structural variants. Furthermore, long-read sequencing of native DNA or RNA molecules reduces amplification bias and preserves base modifications, such as methylation status. Long-read sequencing technologies useful herein can include any suitable long-read sequencing methods, including, but not limited to, Pacific Biosciences (PacBio) single-molecule real-time (SMRT) sequencing, Oxford Nanopore Technologies (ONT) nanopore sequencing, and synthetic long-read sequencing approaches, such as linked reads, proximity ligation strategies, and optical mapping. Synthetic long-read approaches comprise assembly of short reads from the same DNA molecule to generate synthetic long reads, and may be used in conjunction with “true” long-read sequencing technologies, such as SMRT and nanopore sequencing methods.

Single-molecule real-time (SMRT) sequencing facilitates direct detection of, e.g., 5-methylcytosine and 5-hydroxymethylcytosine as well as unmodified cytosine (Weirather JL, et al., “Comprehensive comparison of Pacific Biosciences and Oxford Nanopore Technologies and their applications to transcriptome analysis,” F1000Research, 6:100, 2017). Whereas next-generation sequencing methods detect augmented signals from a clonal population of amplified DNA fragments, SMRT sequencing captures a single DNA molecule, maintaining base modification during sequencing. The error rate of raw PacBio SMRT sequencing-generated data is about 13-15%, as the signal-to-noise ratio from single DNA molecules not high. To increase accuracy, this platform uses a circular DNA template by ligating hairpin adaptors to both ends of target double-stranded DNA. As the polymerase repeatedly traverses and replicates the circular molecule, the DNA template is sequenced multiple times to generate a continuous long read (CLR). The CLR can be split into multiple reads (“subreads”) by removing adapter sequences, and multiple subreads generate circular consensus sequence (“CCS”) reads with higher accuracy. The average length of a CLR is >10 kb and up to 60 kb, with length depending on the polymerase lifetime. Thus, the length and accuracy of CCS reads depends on the fragment sizes. PacBio sequencing has been utilized for genome (e.g., de novo assembly, detection of structural variants and haplotyping) and transcriptome (e.g., gene isoform reconstruction and novel gene/isoform discovery) studies.

ONT is a nanopore-based single molecule sequencing technology (Weirather JL, et al., F1000Research, 6:100, 2017). ONT directly sequences a native single-stranded DNA (ssDNA) molecule by measuring characteristic current changes as the bases are threaded through the nanopore by a molecular motor protein. ONT uses a hairpin library structure similar to the PacBio circular DNA template: the DNA template and its complement are bound by a hairpin adaptor. Therefore, the DNA template passes through the nanopore, followed by a hairpin and finally the complement. The raw read can be split into two “1D” reads (“template” and “complement”) by removing the adaptor. The consensus sequence of two “1D” reads is a “2D” read with a higher accuracy.

5-letter and 6-letter sequencing methods include whole genome sequencing methods capable of sequencing A, C, T, and G in addition to 5mC and 5hmC to provide a 5-letter (A, C, T, G, and either 5mC or 5hmC) or 6-letter (A, C, T, G, 5mC, and 5hmC) digital readout in a single workflow. The processing of the DNA sample is entirely enzymatic and avoids the DNA degradation and genome coverage biases of bisulfite treatment. In an exemplary 5-letter sequencing method developed by Cambridge Epigenetix, the sample DNA is first fragmented via sonication and then ligated to short, synthetic DNA hairpin adaptors at both ends (Füllgrabe, et al. 2022, bioRxiv doi:https://doi.org/10.1101/2022.07.08.499285). The construct is then split to separate the sense and antisense sample strands. For each original sample strand a complementary copy strand is synthesized by DNA polymerase extension of the 3′-end to generate a hairpin construct with the original sample DNA strand connected to its complementary strand, lacking epigenetic modifications, via a synthetic loop. Sequencing adapters are then ligated to the end. Modified cytosines are enzymatically protected. The unprotected Cs are then deaminated to uracil, which is subsequently read as thymine. In any such embodiments, amplification methods may comprise uracil- and/or dihydrouracil-tolerant amplification methods, such as PCR using a uracil-and/or dihydrouracil-tolerant DNA polymerase (i.e., a DNA polymerase that can read and amplify templates comprising uracil and/or dihydrouracil bases). The deaminated constructs are no longer fully complementary and have substantially reduced duplex stability, thus the hairpins can be readily opened and amplified by PCR. The constructs can be sequenced in paired-end format whereby read 1 (P1 primed) is the original stand and read 2 (P2 primed) is the copy stand. The read data is pairwise aligned so read 1 is aligned to its complementary read 2. Cognate residues from both reads are computationally resolved to produce a single genetic or epigenetic letter. Pairings of cognate bases that differ from the permissible five are the result of incomplete fidelity at some stage(s) comprising sample preparation, amplification, or erroneous base calling during sequencing. As these errors occur independently to cognate bases on each strand, substitutions result in a non-permissible pair. Non-permissible pairs are masked (marked as N) within the resolved read and the read itself is retained, leading to minimal information loss and high accuracy at read-level. The resolved read is aligned to the reference genome. Genetic variants and methylation counts are produced by read-counting at base-level.

5hmC has been shown to have value as a marker of biological states and disease which includes early cancer detection from cell-free DNA. In adapting 5-letter to 6-letter sequencing, 5mC is disambiguated from 5hmC without compromising genetic base calling within the same sample fragment. The first three steps of the workflow are identical to 5-letter sequencing described above, to generate the adapter ligated sample fragment with the synthetic copy strand. Methylation at 5mC is enzymatically copied across the CpG unit to the C on the copy strand, whilst 5hmC is enzymatically protected from such a copy. Thus, unmodified C, 5mC and 5hmC in each of the original CpG units are distinguished by unique 2-base combinations. The unmodified cytosines are then deaminated to uracil, which is subsequently read as thymine. The DNA is subjected to PCR amplification and sequencing as described earlier. The reads are pairwise aligned and resolved using a 2-base code. Each of unmodified C, 5mC, and 5hmC can be resolved as the three CpG units are distinct sequencing environments of the 2-base code.

In some embodiments, sequence coverage of the genome may be, for example, less than 5%, 10%, 15%, 20%, 25%, 30%, 40%, 50%, 60%, 70%, 80%, 90%, 95%, 99%, 99.9% or 100%. In some embodiments, the sequence reactions may provide for sequence coverage of, for example, at least 5%, 10%, 15%, 20%, 25%, 30%, 40%, 50%, 60%, 70%, or 80% of the genome. Sequence coverage can be performed on, for example, at least 5, 10, 20, 70, 100, 200 or 500 different genes, or up to, for example, 5000, 2500, 1000, 500 or 100 different genes.

Simultaneous sequencing reactions may be performed using multiplex sequencing. In some embodiments, cell-free nucleic acids may be sequenced with at least, for example, 1000, 2000, 3000, 4000, 5000, 6000, 7000, 8000, 9000, 10000, 50000, or 100,000 sequencing reactions. In other embodiments, cell-free nucleic acids may be sequenced with less than, for example, 1000, 2000, 3000, 4000, 5000, 6000, 7000, 8000, 9000, 10000, 50000, or 100,000 sequencing reactions. Sequencing reactions may be performed sequentially or simultaneously. Subsequent data analysis may be performed on all or part of the sequencing reactions. In some embodiments, data analysis may be performed on at least, for example, 1000, 2000, 3000, 4000, 5000, 6000, 7000, 8000, 9000, 10000, 50000, or 100,000 sequencing reactions. In other embodiments, data analysis may be performed on less than, for example, 1000, 2000, 3000, 4000, 5000, 6000, 7000, 8000, 9000, 10000, 50000, or 100,000 sequencing reactions. An exemplary read depth is 1000-50000 or 1000-10000 or 1000-20000 reads per locus (base).

In general, sequencing of epigenetic target regions, e.g., to analyse a methylation profile of DNA, requires a lesser depth of sequencing than sequencing of a sequence-variable target region, e.g., for analysis of mutations. Hence, lesser sequencing depths, as described herein, may in some cases be adequate for the methods described herein.

### EXAMPLES

### Example 1—Amplicon Sequencing, Anchored Workflows

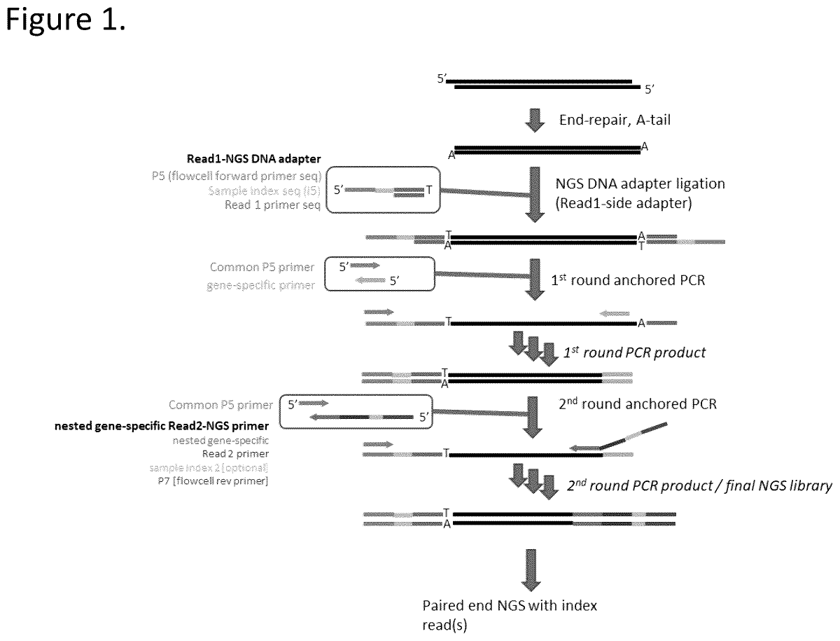

Described herein are methods related to amplicon sequencing, specifically, methods and compositions that utilized anchored multiplex NGS workflows. Generally, amplicon sequencing methods (e.g., AMP-seq) include hybridizing a target nucleic acid molecule with a population of tailed random primers, extending the hybridized tailed random primer using the portion of the target nucleic acid molecule downstream of the site of hybridization as a template, followed by amplifying a portion of the target nucleic acid molecule and the tailed random primer sequence with a first tail primer and a first target-specific primer, amplifying a portion of the amplicon with a second tail primer and a second target-specific primer, sequencing the amplified portion from using a first and second sequencing primer. An example is shown in FIG. 1. However, this sequencing primer can be long (e.g. 80 bp) when only a 20-30 bp of the 80 bp primer is unique for a patient. Yet, amplicon sequencing techniques such as a standard AMP-seq workflow involves use of the full 80 bp primer for every patient+variant combination. Here, a nested gene-specific adapter primer is truncated, yet contains a partial Read2 primer sequence (short, e.g. 4 bases containing at least uracial) that with enzymatic treatment for uracil excision provides an overhang compatible with the subsequent sequencing adapter, which is shorter as not requiring the nest gene-specific portion in conventional amplifon sequencing, relying instead on overhang complementarity instead for a shorter adapter.

### Example 2 —Read2 Overhang dsDNA Adapter Ligation

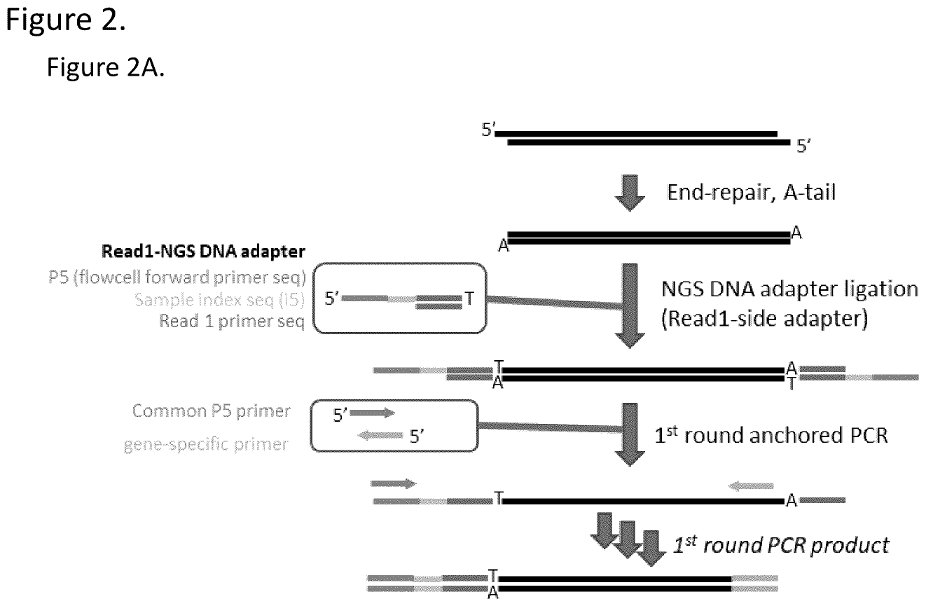

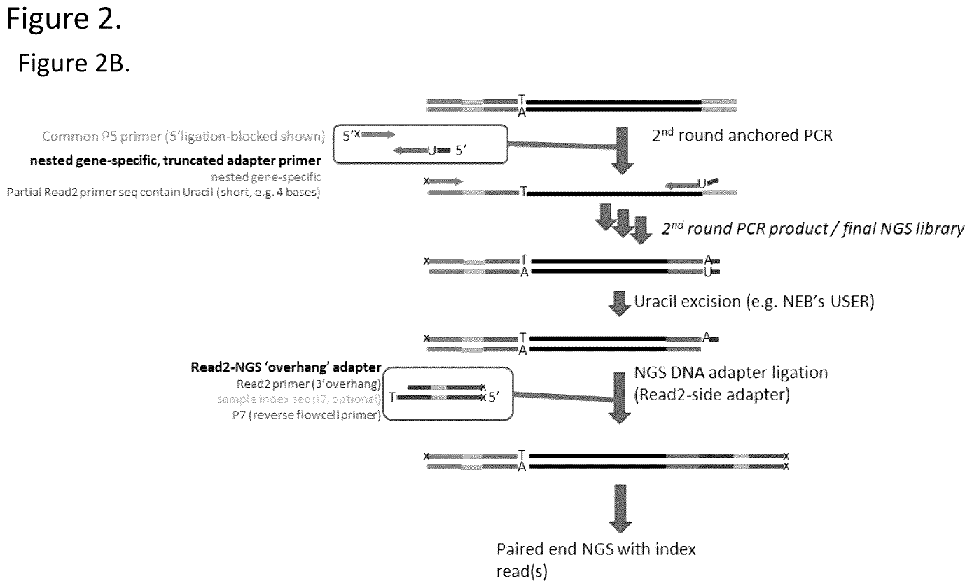

In accordance with the methods and compositions described herein, shown in FIG. 2 is an example of Read2 overhang dsDNA adapter ligation. Here, for example dsDNA Read2-adapter ligation, with oligo end blocking to mitigate undesired ligation events.

### Example 3—Read2 Overhang dsDNA Adapter Ligation

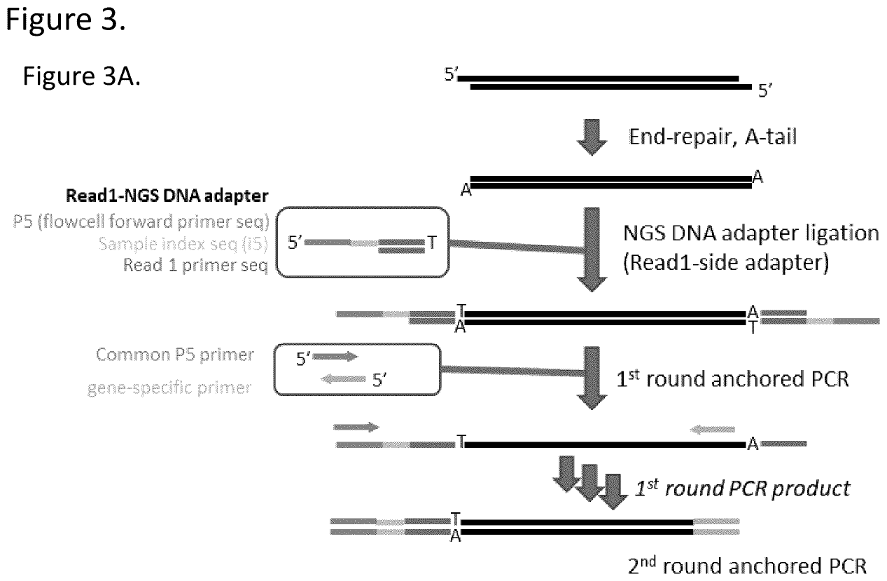

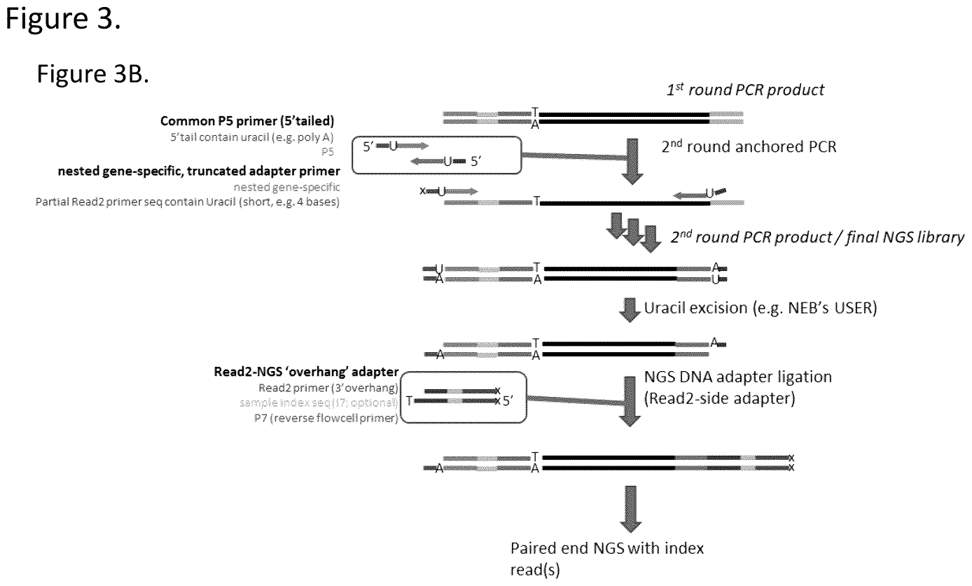

In accordance with the methods and compositions described herein, shown in FIG. 3 is another example of Read2 overhang dsDNA adapter ligation includes dsDNA Read2-adapter ligation, but instead, a P5-primer overhang is generated to mitigate undesired ligation events.

### Example 4—Truncated Read1-Adapter Ligation

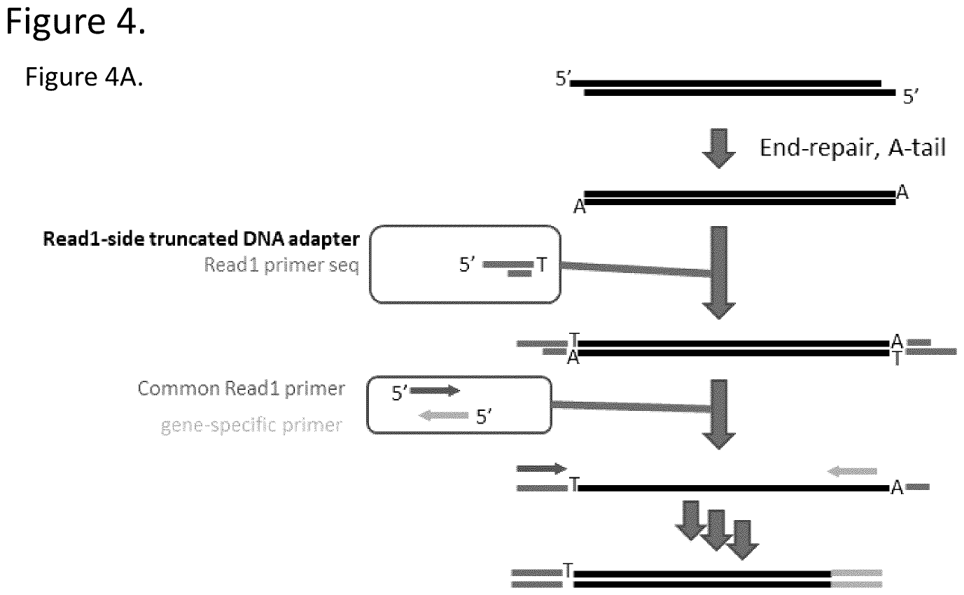

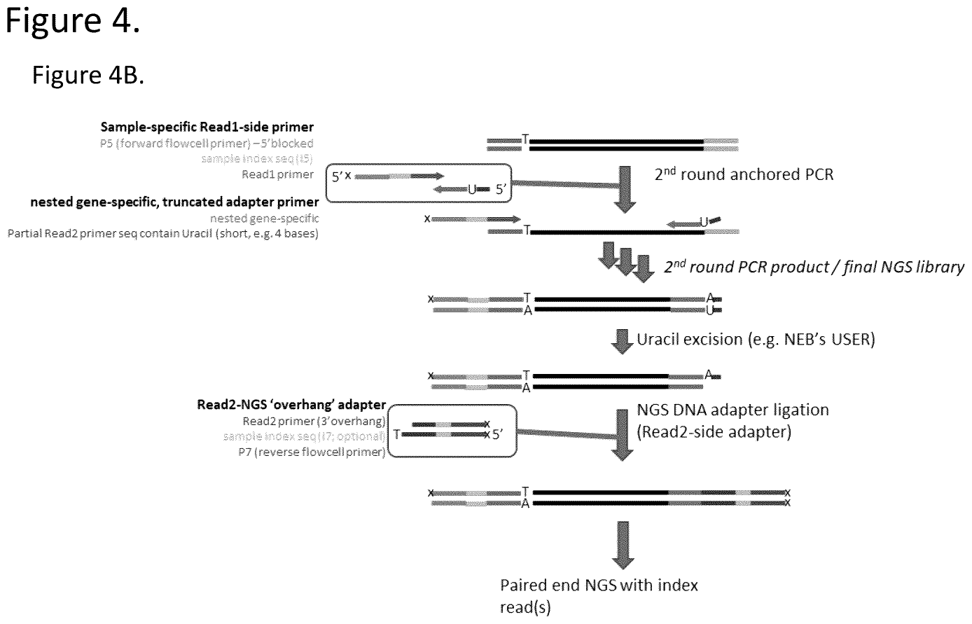

In accordance with the methods and compositions described herein, shown in FIG. 4, it is appreciated that the method can be applied flexibly, with another example here depicting truncated Read1-adapter ligation variant compatible with all workflows described herein.

### Example 5—Read1- and Read2-Overhang Adapter Workflow

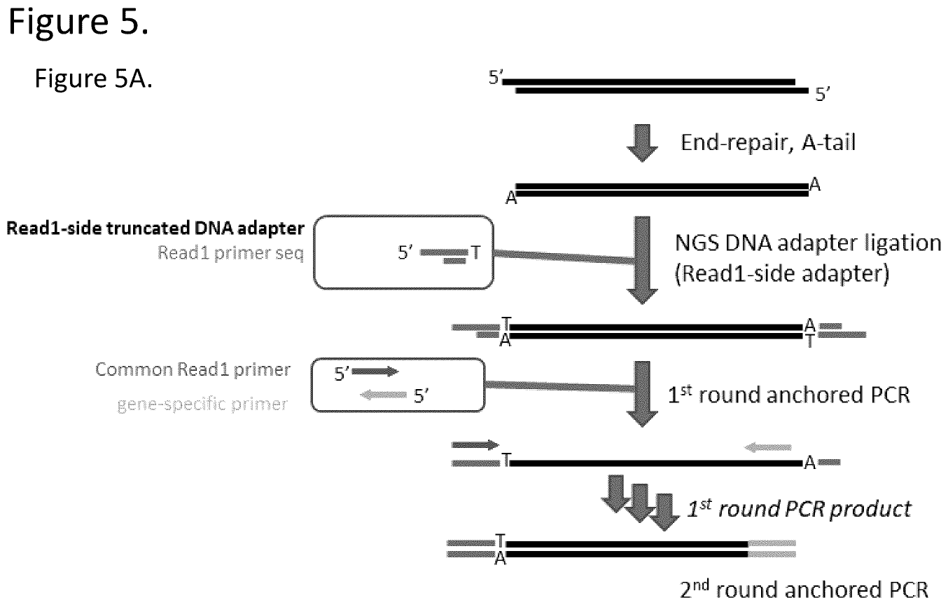

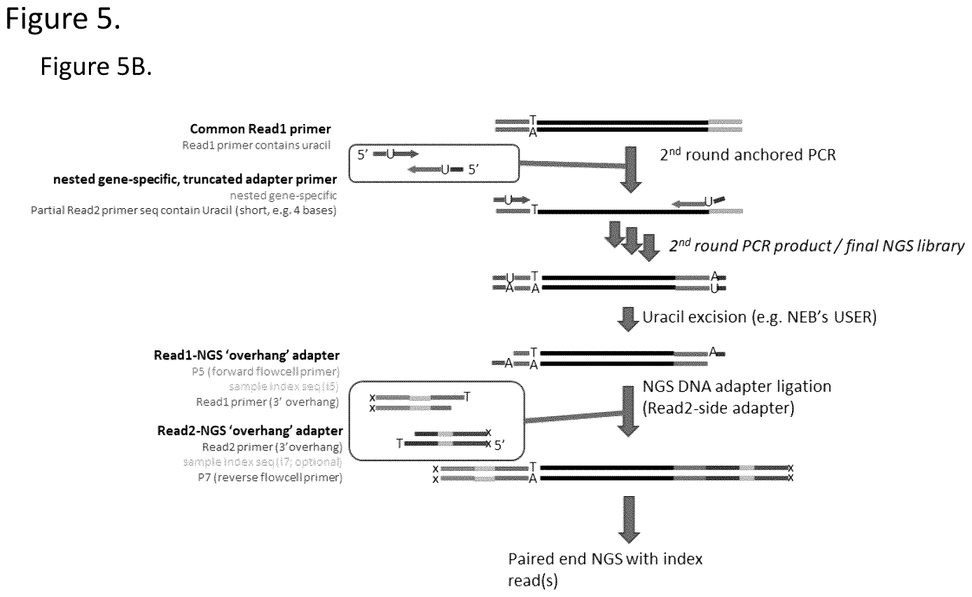

In further accordance with the methods and compositions described herein, shown in FIG. 5 Read1- and Read2-overhang adapter workflow, this approach involves double stranded DNA (dsDNA), with overhangs that target asymmetric sequence ends of Read1- and Read2-primers

### Example 6—Read1- and Read2-Splint Adapter Workflow

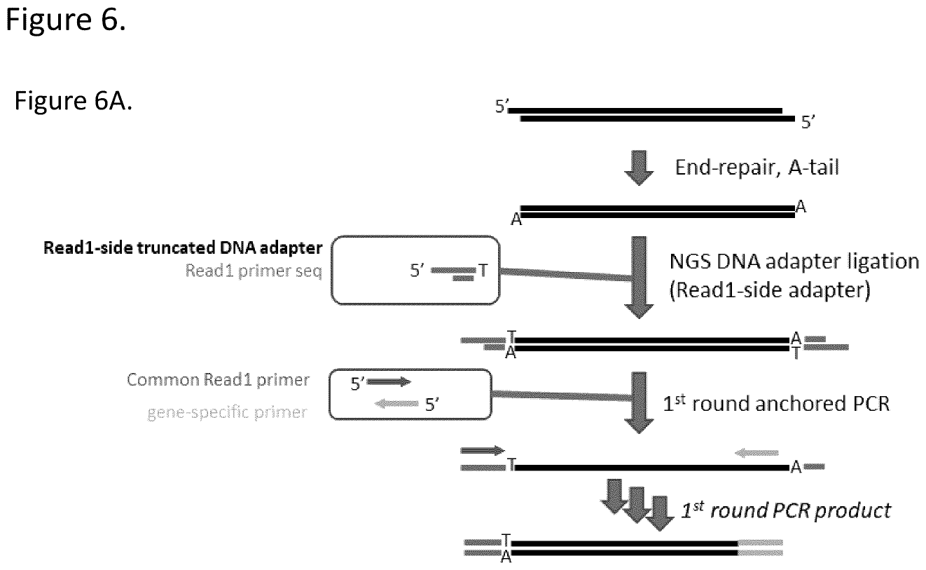

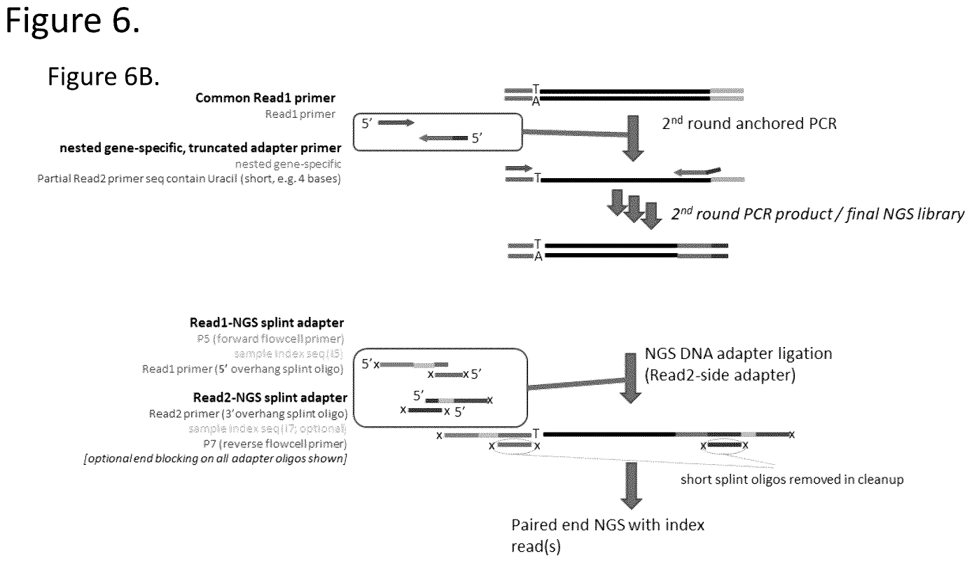

In further accordance with the methods and compositions described herein, shown in FIG. 6 is a Read1- and Read2-splint adapter workflow. Here, overhangs are utilized to target asymmetric sequence ends of Read1- and Read2-primers

### Example 7—Common Read2-Primer Extension

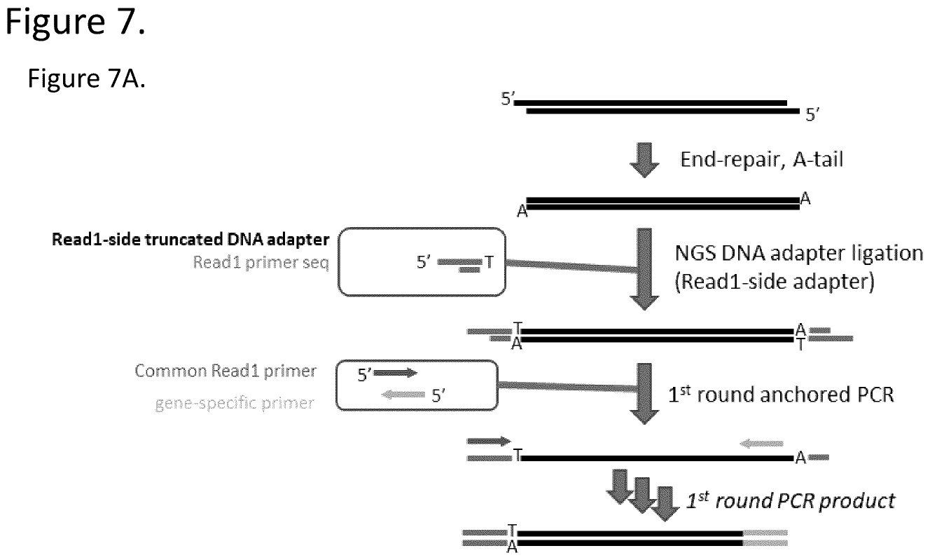

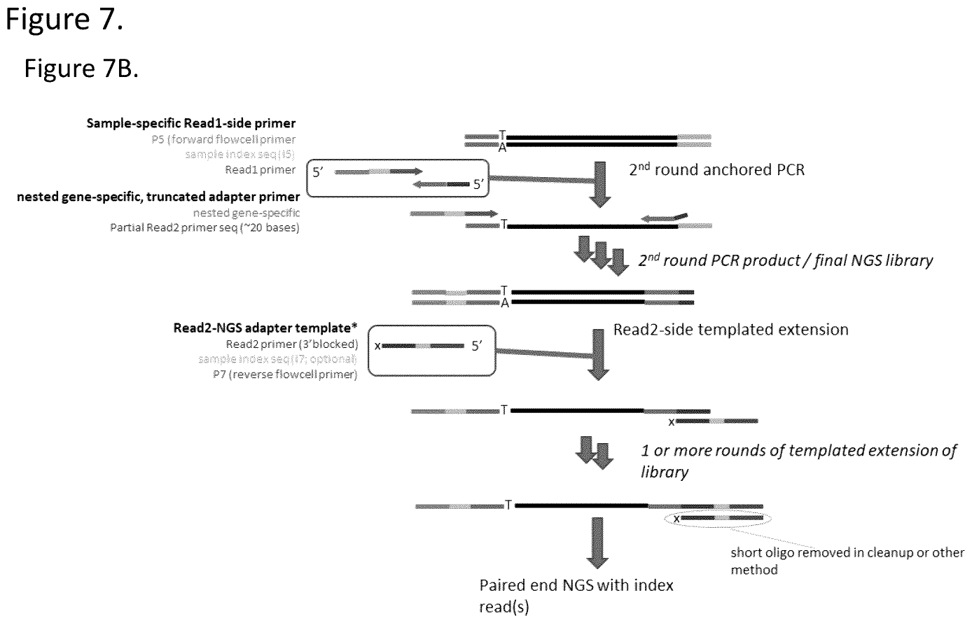

In further accordance with the methods and compositions described herein, shown in FIG. 7 is a common Read2-primer extension, compatible with full and truncated Read1-side adapter ligation workflow, templated extension can be cycled to give multiple extensions per template molecule—requiring lower template oligo than PCR.

### Example 8—Long Primer Manufacture from Gene-Specific Custom Primers

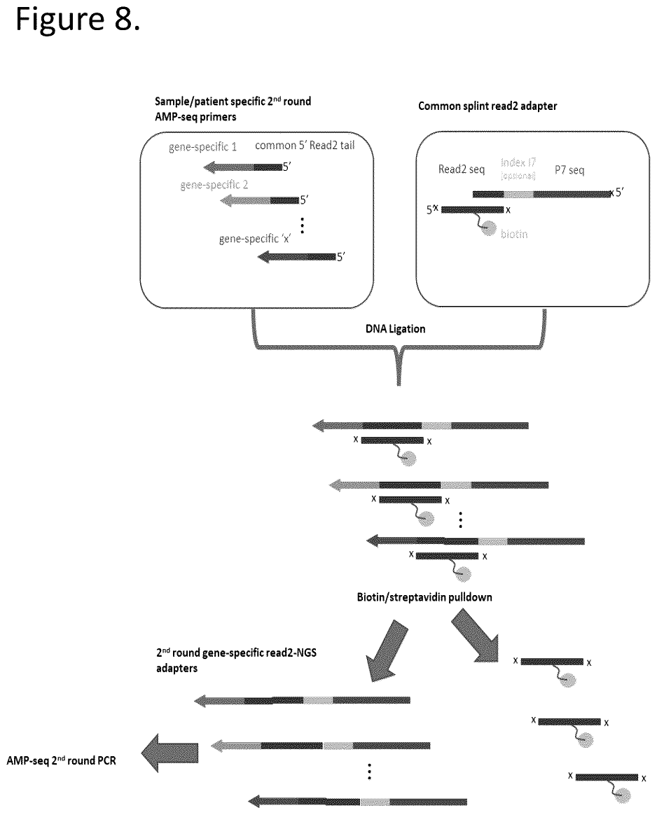

As an additional example related to methods and compositions described herein, shown in FIG. 8 is reducing length of custom primers through use of enzymatic steps to generate long gene-specific adapter primers needed for AMP-seq
Here a standard AMP-seq assay can be used, and the ‘custom long primers’ are pre-manufactured before introduction into assay
One pre-manufacture process would involve
   For each patient/bespoke AMP-seq reaction, order ‘x’ 2nd round gene-specific primers with common 5′ tail—partial sequence of the read2-primer
   Pool the gene-specific custom primers to be used in a multiplex amp reaction into a ligation reaction with splint adapter that can be immobilized/removed (here shown with biotin modification)
   Remove ligation product, gene-specific primers with Read2-NGS tail and take for use in AMP-seq nested/2^{nd} PCR step.
   Note on variants:
      (1) biotinylated splint oligo maybe re-used in manufacturing after removal of ligation product
      (2) individual gene-specific primer ligation may be performed before pooling if desirable for QC
      (3) alternatives to biotin/streptavidin removal of splint oligo include:
         (a) unique placement of 5′ phosphate group on splint oligo and targeting for degradation by lambda endonuclease post-ligation
         (b) uracil use in splint oligo, enabling targeting for degradation post-ligation with uracil-removal means

1. A method of determining the nucleotide sequence contiguous to a known target nucleotide sequence, the method comprising;
hybridizing a target nucleic acid molecule comprising the known target nucleotide sequence with a population of tailed random primers;
extension of a hybridized tailed random primer using the portion of the target nucleic acid molecule downstream of the site of hybridization as a template; and
amplifying a portion of the target nucleic acid molecule and the tailed random primer sequence with a first tail primer and a first target-specific primer.

2. The method of claim 1, comprising sequencing the amplified using a first and second sequencing primer.

3. The method of claim 1, wherein the population of tailed random primers each comprise a 5′ nucleic acid sequence that is identical to a first sequencing primer.

4. The method of claim 1, wherein the first tail primer comprises a nucleic acid sequence identical to the 5′ portion of the tailed random primer.

5. The method of claim 1, wherein a second tail primer comprises a nucleic acid sequence identical to a portion of a first sequencing primer.

6. The method of claim 1, wherein each tailed random primer in the population of tailed random primers further comprises a spacer nucleic acid sequence between the 5′ nucleic acid sequence identical or complementary to a first sequencing primer and the 3′ nucleic acid sequence comprising about 6 to about 12 random nucleotides.

7. The method of claim 1, wherein unhybridized primers are removed from the reaction after an extension step.

8. The method of claim 1, wherein the second tail primer is nested with respect to the first tail primer by at least 3 nucleotides.

9. The method of claim 1, wherein the first target-specific primer further comprises a 5′ tag sequence portion comprising a nucleic acid sequence of high GC content which is not substantially complementary to or substantially identical to any other portion of any of the primers.

10. The method of claim 1, wherein a second tail primer is identical to a full-length first sequencing primer.

11. The method of claim 1, comprising sequencing a nucleic acid product by a next-generation sequencing method.

12. The method of claim 11, wherein a first and second sequencing primers are compatible with the selected next-generation sequencing method.

13. The method of claim 1, wherein the method comprises contacting the sample, or separate portions of the sample, with a plurality of sets of first and second target-specific primers.

14. The method of claim 1, wherein the method comprises contacting a single reaction mixture comprising the sample with a plurality of sets of first and second target-specific primers.

15. The method of claim 14, wherein the plurality of sets of first and second target-specific primers specifically anneal to known target nucleotide sequences comprised by separate genes.

16. The method of claim 1, wherein at least two sets of first and second target-specific primers specifically anneal to different portions of a known target nucleotide sequence.

17. The method of claim 1, wherein at least two sets of first and second target-specific primers specifically anneal to different portions of a single gene comprising a known target nucleotide sequence.

18. The method of claim 1, wherein at least two sets of first and second target-specific primers specifically anneal to different exons of a gene comprising a known nucleotide target sequence.

19. The method of claim 1, comprising a plurality of first target-specific primers, each first target-specific primer comprising identical 5′ tag sequence portions.

20. The method of claim 1, comprising first and/or second target specific primers that are truncated.

21. The method of claim 1, comprising first and/or second target specific primers comprise uracil and/or a modified base.

22-23. (canceled)

24. The method of claim 1, comprising first and/or second sequencing primers each comprising a sequence at least partially complementary to the first and/or second target specific primers.

25. The method of claim 1, comprising uracil excision.

26. The method of claim 1, comprising template extension.
